SEQUENCE LISTING (2 sequences)

SEQ ID NO: 1 - protein, 552 aa, synthetic construct
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 2 - protein, 11 aa, synthetic construct
DGYVDPSGGDR

(19) United States
(12) Patent Application Publication
(10) Pub. No.: US 2023/0201173 A1
(43) Pub. Date: Jun. 29, 2023

(54) SMAD4 Complex Inducers and Uses in Cancer Therapy

(71) Applicant: Emory University, Atlanta, GA (US)

(72) Inventors: Haian Fu, Decatur, GA (US); Xiulei Mo, Atlanta, GA (US); Cong Tang, Lisbon (PT)

(21) Appl. No.: 18/065,219

(22) Filed: Dec. 13, 2022

Related U.S. Application Data
(60) Provisional application No. 63/265,321, filed on Dec. 13, 2021.

(51) Int. Cl. A61K 31/437; A61K 31/404; A61K 31/4045; A61P 35/00; G01N 33/68

(52) U.S. Cl. CPC A61K 31/437; A61K 31/404; A61K 31/4045; A61P 35/00; G01N 33/6845

(57) ABSTRACT

In certain embodiments, this disclosure relates to methods of identifying cancer agents and treating cancer with identified agents. In certain embodiments, the caner agents are capable of inducing or stabilizing SMAD4 oligomerization.

### CROSS-REFERENCE TO RELATED APPLICATIONS

This application claims the benefit of U.S. Provisional Application No. 63/265,321 filed Dec. 13, 2021. The entirety of this application is hereby incorporated by reference for all purposes.

### STATEMENT REGARDING FEDERALLY SPONSORED RESEARCH OR DEVELOPMENT

This invention was made with government support under CA217875 awarded by the National Institutes of Health. The government has certain rights in this invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 20174US.xml. The XML file is 4 KB, was created on Dec. 12, 2022, and is being submitted electronically via the USPTO patent electronic filing system.

### BACKGROUND

Conventional chemotherapy drugs used for treating solid tumors are not universally effective and recurrence is common. Thus, there is a need to identify improved therapies. SMAD4 gene mutations are observed in many pancreatic and colon tumors. SMAD4 is a protein that binds to DNA and recognizes a sequence called the Smad-binding element (SBE). SMAD4 acts as a tumor suppressor and a central mediator of the TGF-beta pathway. SMAD4 contains a MH2 domain that is involved in SMAD protein homo- and hetero-oligomerization, e.g., with other SMAD proteins such as SMAD2 and SMAD3.

Fleming et al. report a SMAD4 hotspot region within the MH2 domain spanning Asp351-Pro356 and Arg361 forms key interactions that interface with SMAD2 or SMAD3. Cancer Res, 2013, 73 (2): 725-735. See also. Zhao et al., The role of TGF-β3/SMAD4 signaling in cancer. Int. J. Biol. Sci. 2018, 14(2): 111-123.

Newton et al. report methods for determination of the affinity of protein:protein interactions in time resolved fluorescence assays. Journal of Biomolecular Screening, 2008, 13(7):674-682.

Zhou et al. report compounds that facilitate and/or induce Fas-mediated apoptosis. U.S. Pat. No. 6,284,783 (2001).

References reported herein are not an admission of prior art.

### SUMMARY

In certain embodiments, this disclosure relates to methods of identifying cancer agents and treating cancer with identified agents, e.g., 3,4-di(indol-3-yl)-pyrrole-2,5-dione derivatives disclosed herein. In certain embodiments, this disclosure relates to methods of screening a test compound for the ability to induce SMAD4 and another SMAD protein to interact. In certain embodiments, the caner agents are capable of inducing or stabilizing SMAD4 oligomerization, i.e., hetero-oligomerization with, e.g., SMAD2 and/or SMAD3, or homo-oligomerization.

In certain embodiments, this disclosure relates to methods of treating cancer by administering an effective amount of a compound disclosed herein to a subject in need thereof. In certain embodiments, the subject is diagnosed with cancer and has a SMAD4 gene mutation. In certain embodiments, this disclosure relates to methods of diagnosing and treating cancer comprising obtaining a sample from a subject and detecting a mutation of tumor suppressor gene SMAD4 in the sample; and administering an effective amount of an agent identified using methods disclosed herein to the subject in need thereof.

In certain embodiments, this disclosure relates to methods of treating cancer comprising administering an effective amount of a compound having a 3,4-di(indol-3-yl)-pyrrole-2,5-dione backbone structure, or salt thereof, to the subject in need thereof. In certain embodiments, the compound is 3-(3-(4-(1-methyl-1H-indol-3-yl)-2,5-dioxo-2,5-dihydro-1H-pyrrol-3-yl)-1H-indol-1-yl)propyl carbamimidothioate (Ro-31-8220), salt, or derivative thereof. In certain embodiments, the compound is 3-(1-(3-(dimethylamino)propyl)-5-methoxy-1H-indol-3 -yl)-4-(1H-indol-3 -yl)-1H-pyrrole-2,5-dione (Go-6983), salt, or derivative thereof.

In certain embodiments, this disclosure relates to methods screening test compounds to identify agents that compounds are capable of inducing or stabilizing SMAD4 complexing with SMAD2 and/or SMAD3.

In certain embodiments, this disclosure relates to pharmaceutical compositions comprising compounds disclosed herein and optionally a pharmaceutically acceptable excipient, and kits related thereto.

### BRIEF DESCRIPTION OF THE SEVERAL VIEWS OF THE DRAWINGS

FIG. 1A shows distribution (top) and count (bottom) of SMAD4 missense mutation in colon cancer. Inactivating SMAD4 mutations have been recurrently identified in colon (14%), and pancreatic cancer (16%), stomach cancer (7.5%), esophageal cancer (6.6%), cervical cancer (4.4%) and lung cancer (4.4%). In colon cancer, the SMAD4 R361H is the most frequently occurred mutation with 14% of all the SMAD4 missense mutations. The SMAD4 R361H is found in cervical cancer (16%), pancreatic cancer (3%), lung cancer (4.5%) and breast cancer (10%) among all the SMAD4 mutated patient samples.

FIG. 1B illustrates a TR-FRET assay for monitoring the SMAD4/SMAD3 protein-protein interaction (PPI) to discover small-molecule PPI inducers. Anti-Flag-Tb coupled with Flag-SMAD3 serves as the TR-FRET donor and anti-His-D2 coupled with His-SMAD4R361H serves as the acceptor. At the basal level, R361H impairs SMAD4 interaction with SMAD3, yielding low TR-FRET signal. Upon treatment with a PPI inducer, the induced SMAD3/SMAD4R361H complex formation brings two fluorophores into close proximity (<10 nm), generating a high TR-FRET signal.

FIG. 1C shows data on dose-dependent TR-FRET signals of the SMAD4WT/SMAD3 PPI. The cell lysate from the HEK293T cells expressing the His-SMAD4WT and Flag-SMAD3 or the empty vector controls were serial diluted as indicated.

FIG. 1D shows data on dynamic assay windows as defined by the signal-to-background ratio (S/B) of the dose-response TR-FRET signals. The cell lysates from the HEK293T cells expressing His-SMAD4WT or SMAD4R361H together with Flag-SMAD3 were serial diluted for TR-FRET assay.

FIG. 1E shows a waterfall plot showing the change of TR-FRET signal (ΔTR-FRET) induced by compounds from the primary screening. The data are presented as the percentage of the DMSO control from the primary screening. Chemical structures of two highly ranked SMAD4R361H/SMAD3 PPI inducers, Ro-31-8220 and Go-6983.

FIG. 1F shows dose-dependent curves of (right) Ro-31-8220 and (left) Go-6983 in enhancing the TR-FRET signal of the SMAD4R361H/SMAD3 PPI.

FIG. 2 shows a table with data on bisindolylmaleimide derivatives. EC5o were calculated from the TR-FRET dose-response assay. Shown are activities in enhancing the SMAD4R361H-SMAD3 PPI. From the dose-response study using the TR-FRET assay, all the bisindolylmaleimide derivatives exhibited similar PPI potentiation effects, with EC₅₀ values ranging from 4 to 20 μM, whereas the other two structurally diverse PKC inhibitors had no significant effect up to 100 μm,M. Ro-31-8220 has the chemical name 3-(3-(4-(1-methyl-1H-indol-3-yl)-2,5-dioxo-2,5-dihydro-1H-pyrrol-3-yl)-1H-indol-1-yl)propyl carbamimidothioate. Go-6983 has the chemical name 3-(1-(3-(dimethylamino)propyl)-5-methoxy-1H-indol-3-yl)-4-(1H-indol-3-yl)-1H-pyrrole-2,-dione.

FIG. 3A shows bar graphs indicating the quantification of colon cancer cells in G1/S phase. Parental HCT116 (WT) and isogenic R361H cells were treated with TGF-β (10 ng/mL), Ro-31-8220 (1 μM), Go-6983 (10 μM), or in combination as indicated. The percentage of cells staying in G1/S phase was determined by flow cytometry.

FIG. 3B shows data indicating a synergistic effect of TGF-β with Ro-31-8220 or Go-6983 in inhibiting cell viability in the set of isogenic HCT116 colon cancer cells. HCT116 cells (WT) and corresponding SMAD4 knockout (null) or R361H, isogenic cells were treated with TGF-β (10 ng/mL), Ro-31-8220 (1 μM), Go-6983 (10 μM), or in combination as indicated. The cell viability after treatment was measured using the CellTiter-Blue™ reagent and normalized to the untreated vehicle or DMSO controls (% C).

FIG. 3C shows data from images indicating a synergistic effect of TGF-β and Ro-31-8220 or Go-6983 in inhibiting colon cancer cell colony formation. Parental HCT116 (WT) and isogenic R361H cells were treated with TGF-β (10 ng/mL), Ro-31-8220 (1 μM), Go-6983 (10 μM), or in combination as indicated. Bar graphs of the quantification of colony area from the colony formation assay showing the synergistic effect of TGF-β with Ro-31-8220 or Go-6983 in inhibiting colon cancer cell colony formation.

### DETAILED DESCRIPTION

Before the present disclosure is described in greater detail, it is to be understood that this disclosure is not limited to embodiments described, and as such may, of course, vary. It is also to be understood that the terminology used herein is for the purpose of describing embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

“SMAD4” or “Mothers Against Decapentaplegic homolog 4” refers to a protein that binds to DNA and recognizes a sequence called the Smad-binding element (SBE). SMAD4 acts as a tumor suppressor and a central mediator of the TGF-beta pathway. SMAD4 contains a MH2 domain that is involved in SMAD protein oligomerization, e.g., with other SMAD proteins such as SMAD2 and SMAD3. Homo sapiens SMAD family member 4 (SMAD4), transcript variant 2, mRNA is NCBI Reference Sequence: NM 001407041.1. Alternative codons for expressing amino acid mutations are in reference to positions relative to a protein having the following amino acid sequence (or natural variants):

MDNMSITNTPTSNDACLSIVHSLMCHRQGGESETFAKRAIESLVKKLKEKKDELD SLITAITTNGAHPSKCVTIQRTLDGRLQVAGRKGFPHVIYARLWRWPDLHKNELKHVKY CQYAFDLKCDSVCVNPYHYERVVSPGIDLSGLTLQSNAPSSMMVKDEYVHDFEGQPSL STEGHSIQTIQHPPSNRASTETYSTPALLAPSESNATSTANFPNIPVASTSQPASILGGSHSE GLLQIASGPQPGQQQNGFTGQPATYHHNSTTTWTGSRTAPYTPNLPHHQNGHLQHHPP MPPHPGHYWPVHNELAFQPPISNHPAPEYWCSIAYFEMDVQVGETFKVPSSCPIVTVDG YVDPSGGDRFCLGQLSNVHRTEAIERARLHIGKGVQLECKGEGDVWVRCLSDHAVFVQ SYYLDREAGRAPGDAVHKIYPSAYIKVFDLRQCHRQMQQQAATAQAAAAAQAAAVA GNIPGPGSVGGIAPAISLSAAAGIGVDDLRRLCILRMSFVKGWGPDYPRQSIKETPCWIEI HLHRALQLLDEVLHTMPIADPQPLD (SEQ ID NO: 1), wherein the embedded sequence DGYVDPSGGDR (SEQ ID NO: 2) refers to amino acid positions 351 to 361 (in bold).

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

The term “effective amount” refers to that amount of a compound or pharmaceutical composition described herein that is sufficient to effect the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

The terms “protein,” “peptide,” and “polypeptide” refer to compounds comprising amino acids joined via peptide bonds and are used interchangeably. As used herein, where “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit be limited to natural amino acids. The term includes non-naturally occurring amino acids and modifications such as, substitutions, glycosylations, and addition of hydrophilic or lipophilic moieties.

In the context of a fusion or chimeric peptide (a peptide comprising two or more peptide segments), a “heterologous” peptide sequence is a comparative term and refers to a peptide segment that would not naturally occur together with the other segment, e.g., because one the of the segments is derived from a different organism, a label, or random. In certain embodiments, a heterologous fusion peptide of this disclosure may contain a peptide sequence disclosed herein and a fluorescent protein sequence, a protease cleaving sequence, a self-cleaving sequence, a ligand, antibody epitope, or a polyhistidine sequence.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon to carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

As used herein, the term “small molecule” refers to any variety of covalently bound molecules with a molecular weight of less than 900 or 1000. Typically, the majority of atoms include carbon, hydrogen, oxygen, nitrogen, and to a lesser extent sulfur and/or a halogen. Examples include steroids, short peptides, mono or polycyclic aromatic or non-aromatic, heterocyclic compounds.

As used herein, the term “combination with” when used to describe administration with an additional treatment means that the agent may be administered prior to, together with, or after the additional treatment, or a combination thereof.

A “chemotherapy agent,” “chemotherapeutic,” “anti-cancer agent,” or the like, refer to molecules that are recognized to aid in the treatment of a cancer.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O-—, or vice versa. The derivative may be a prodrug. A derivative may be a polypeptide variant. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters of carboxylic acids, e.g., ethyl esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide and benzamide derivatives of an amine functional group in the active compound and the like.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, alkanoyl, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O )ORb, —NRaSO2Rb, —C(═O)Ra, —C(═O )ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkanoyl, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3 -methyl- 1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkanoyl” refers to an alkyl as defined above with the indicated number of carbon atoms attached through a carbonyl bride (i.e., —(C═O)alkyl).

Fluorescence Resonance Energy Transfer (FRET) is a physical phenomenon when different molecules transfer (absorb or emit) distance-dependent energy from a donor molecule to an acceptor molecule. Pairs of molecules that interact in such a manner are referred to as a “FRET donor/acceptor pairs.” As the donor and acceptor are not the same molecules, FRET can be detected by the appearance of fluorescence of the acceptor or by quenching of donor fluorescence. Examples or FRET donor/acceptor pairs include fluorescent dyes, fluorescent proteins, metal nanoparticles (quantum dots), and lanthanide compounds, such as europium and terbium which are typically complexed with a cryptand (Tb) for purposes of solubility. Cryptands are synthetic, bicyclic and polycyclic, multidentate ligands used to stabilize metal cations in aqueous solutions. Such a fluorescent metallic complex is typically made up of a lanthanide or ruthenium core and a polydentate complexing agent, e.g., comprising at least 2, and preferably between 2 and 9, electron donor heteroatoms, such as N, O or S. These coordination bonds with the lanthanide or the ruthenium. Preferably, the fluorescent metallic complex has one or more chromophores consisting of aromatic structures; preferably, these aromatic structures comprise 1, 2 or 3 heteroatoms chosen from N and O, which act as lanthanide or ruthenium coordination atoms. Many complexing agents have been described. By way of examples of complexing agents, mention may be made of the following compounds: EDTA, DTPA, TTHA, DOTA, NTA, HDTA, DTPP, EDTP, HDTP, NTP, DOTP, DO3A and DOTAGA. Fluorescent metallic complexes are stable in terms of association/dissociation of the complexing agent and of the core element.

The donor and/or acceptor fluorescent agents are generally each conjugated to a pair of binding partners, in accordance with the usual application of the TR-FRET technique to the study of biological phenomena. For example, these agents can each be coupled to an antibody, an enzyme substrate, a peptide, a membrane receptor ligand, etc. The fluorescent agent may be any entity that can fluoresce and/or quench fluorescence. In Fluorescence Resonance Energy Transfer (FRET), energy of an excited fluorophore (sometime referred to as a “donor”) is transferred to a neighboring acceptor molecule, e.g., enhancing, changing, or limiting fluorescence of the donor fluorophore due to quenching (“quencher”). The quencher molecule can be another fluorescent dye or a non-fluorescent dark quencher. The distance between the donor and the acceptor is related to the intensity changes.

The acceptor and donor fluorescent compounds may be chosen from allophycocyanins, rhodamines, cyanines, squaraines, coumarins, proflavines, acridines, fluoresceins, boron-dipyrromethane derivatives and nitrobenzoxadiazole. Examples of fluorescent dyes include fluorescein dyes, fluorescein isothiocyanate (FITC) dyes, rhodamine-based dyes such as tetramethylrhodamine (TMR) dyes, carboxytetramethylrhodamine dyes (TAMRA), 6-(2-carboxyphenyl)-1,11-diethyl-3,4,8,9,10,11-hexahydro-2H-pyrano[3,2-g:5,6-g′]diquinolin-1-ium dyes (ATTO™ dyes) and other such as 4,4-difluoro-4-bora-3a,4a-diaza-s-indacene (BODIPY) dyes, BODIPY FL™, Oregon Green 488™, Rhodamine Green™, Oregon Green 514™, TET™, Cal Gold™, BODIPY R6G™, Yakima Yellow™, JOE™, HEX™, Cal Orange™, BODIPY TMR-X™, Quasar-570/Cy3™, Rhodamine Red-X™, Redmond Red™, BODIPY 581/591™, ROX™, Cal Red/Texas Red™, BODIPY TR-X™, BODIPY 630/665-X™, Pulsar-650™, Quasar-670/Cy5™, Cy3.5™, and Cy5.5™. Examples of quenchers include fluorescein, rhodamine, and cyanine dyes, dabcyl, and Black Hole Quenchers™ (BHQs). Dark quenchers include dabcyl, QSY 35™, BHQ-0™, Eclipse™, BHQ-1™, QSY 7™, QSY 9™, BHQ-2™, ElleQuencher™, Iowa Black™, QSY 21™, BHQ-3™.

The following fluorescent proteins can also be used: cyan fluorescent proteins (AmCyanl, Midori-Ishi Cyan, mTFP1), green fluorescent proteins (EGFP, AcGFP, TurboGFP, Emerald, Azami Green, ZsGreen), yellow fluorescent proteins (EYFP, Topaz, Venus, mCitrine, YPet, PhiYFP, ZsYellow1, mBanana), orange and red fluorescent proteins (Orange kusibari, mOrange, tdtomato, DsRed, DsRed2, DsRed-Express2, DsRed-Monomer, mTangerine, AsRed2, mRFP1, JRed, mCherry, mStrawberry, HcRed1, mRaspberry, HcRed-Tandem, mPlim, AQ143, allophycocyanins, XL665, D2, and proteins which are fluorescent in the far-red range (mKate, mKate2, tdKatushka2) or fragments thereof.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

### Pharmaceutical Uses, Compositions, and Kits

In certain embodiments, this disclosure relates to methods of treating cancer using compounds identified by methods disclosed herein. In certain embodiments, the compounds are capable of inducing and/or stabilizing SMAD4 hetero-oligomerization with SMAD2 and/or SMAD3.

In certain embodiments, this disclosure relates to methods of treating cancer comprising administering an effective amount of a compound disclosed herein to a subject in need thereof. In certain embodiments, the subject is diagnosed with cancer and has a SMAD4 gene mutation.

In certain embodiments, this disclosure relates to methods screening test compounds to identify agents that compounds are capable of inducing or stabilizing SMAD4 complexing with SMAD2 and/or SMAD3.

In certain embodiments, the compounds are capable of inducing or stabilizing DNA binding at the Smad-binding element (SBE).

In certain embodiments, this disclosure relates to methods of diagnosing and treating cancer comprising obtaining a sample from a subject and detecting a mutation of tumor suppressor gene SMAD4 (gene or mRNA expression) in the sample; and administering an effective amount of a compound disclosed herein to the subject in need thereof.

In certain embodiments, this disclosure relates to methods of diagnosing and treating cancer comprising obtaining a sample from a subject and detecting a mutation encoding amino acid position 361 of tumor suppressor gene SMAD4 in the sample; and administering an effective amount of an agent identified as capable of inducing or stabling SMAD4 oligomerization to the subject in need thereof.

In certain embodiments, this disclosure relates to methods of diagnosing and treating cancer comprising obtaining a sample from a subject and detecting a mutation encoding amino acid position 361 of tumor suppressor gene SMAD4 in the sample; and administering an effective amount of a compound having a 3,4-di(indol-3-yl)-pyrrole-2,5-dione backbone structure, or salt thereof, to the subject in need thereof.

In certain embodiments, the mutation encodes amino acid at position 361 of tumor suppressor gene SMAD4. In certain embodiments, the mutation that encodes amino acid position 361 provides a codon mutation that translates histidine (H) instead of arginine (R) at amino acid position 361. In certain embodiments, the mutation that encodes amino acid position 361 provides a codon mutation that translates cysteine (C) instead of arginine (R) at amino acid position 361.

In certain embodiments, the subject is diagnosed with colon cancer, pancreatic cancer, skin cancer, stomach cancer, esophageal cancer, cervical cancer, or lung cancer. In certain embodiments, the compound has a 3,4-di(indol-3-yl)-pyrrole-2,5-dione backbone structure, or salt thereof.

In certain embodiments, the compound is 3-(3-(4-(1-methyl-1H-indok-3-yl)-2,5-dioxo-2,5-dihydro-1H-pyrrol-3-yl)-1H-indol-1-yl)propyl carbamimidothioate (Ro-31-8220), salt, or derivative thereof.

In certain embodiments, the compound is 3-(1-(3-(dimethylamino)propyl)-5-methoxy-1H-indol-3-yl)-4-(1H-indol-3-yl)-1H-pyrrole-2,5-dione (Go-6983), salt, or derivative thereof.

In certain embodiments, the compound is 3-(1-(3-aminopropyl)-1H-indol-3-yl)-4-(1-methyl-1H-indol-3-yl)-1H-pyrrole-2,5-dione (Ro-31-7549), salt, or derivative thereof.

In certain embodiments, the compound is 3-(8-(aminomethyl)-6,7,8,9-tetrahydropyrido[1,2-a]indol-10-yl)-4-(1-methyl-1H-indol-3-yl)-1H-pyrrole-2,5-dione (Ro-31-8425), salt, or derivative thereof.

In certain embodiments, the compound is 3-(8-((dimethylamino)methyl)-6,7,8,9-tetrahydropyrido[1,2-a]indol-10-yl)-4-(1-methyl-1H-indol-3-yl)-1H-pyrrole-2,5-dione (Ro-32-0432), salt, or derivative thereof.

In certain embodiments, this disclosure relates uses of compounds disclosed herein for the treatment of cancer. In certain embodiments, this disclosure relates to the production of a medicament using compounds disclosed herein for the treatment of cancer.

In certain embodiments, a compound disclosed herein is administered in combination with another anticancer agent. Contemplated examples include the following agents or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, rib ociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

In certain embodiments, the chemotherapy agent is an anti-PD-1, anti-PD-L1 anti-CTLA4 antibody or combinations thereof, such as an anti-CTLA4 (e.g., ipilimumab, tremelimumab) and anti-PD1 (e.g., nivolumab, pembrolizumab, cemiplimab) and anti-PD-L1 (e.g., atezolizumab, avelumab, durvalumab).

In certain embodiments, the disclosure relates to a method of treating or preventing cancer comprising administering an effective amount of a pharmaceutical composition comprising compound disclosed herein to a subject in need thereof. In certain embodiments, the subject is diagnosed with or at risk of cancer. In certain embodiments, the compound is administered in combination with a second therapeutic agent. In certain embodiments, the cancer is selected from bladder cancer, lung cancer, breast cancer, melanoma, colon and rectal cancer, non-Hodgkin lymphoma, Burkett lymphoma, endometrial cancer, pancreatic cancer, kidney cancer, prostate cancer, leukemia, thyroid cancer, and brain cancer.

In certain embodiment, this disclosure relates to methods for the treatment a subject at risk of, exhibiting symptoms of, suspected of, or diagnosed with a cancer or neoplasm selected from skin cancer, melanoma, Barret's adenocarcinoma; biliary tract carcinomas; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system), colorectal cancer, including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; and thyroid cancers.

The compounds disclosed herein can be used alone in the treatment of each of the foregoing conditions or can be used to provide additive or potentially synergistic effects with certain existing chemotherapies, radiation, biological or immunotherapeutics (including monoclonal antibodies) and vaccines. Compositions and pharmaceutical compositions comprising a compound as reported herein may be useful for restoring effectiveness of certain existing chemotherapies and radiation and or increasing sensitivity to certain existing chemotherapies and/or radiation.

In certain embodiments, this disclosure relates to pharmaceutical compositions comprising compounds disclosed herein and a pharmaceutically acceptable excipient and kits related thereto. In certain embodiments, a composition comprising a compound or salt thereof as reported herein of the present disclosure can be administered to a subject either alone or as a part of a pharmaceutical composition.

Pharmaceutical compositions typically comprise an effective amount of a compound and a suitable pharmaceutical acceptable excipient or carrier. The preparations can be prepared in a manner known per se, which usually involves mixing the compounds according to the disclosure with the one or more pharmaceutically acceptable carriers, and, if desired, in combination with other pharmaceutical active compounds under aseptic conditions. Pharmaceutically acceptable salts, solvates, and hydrates of the compounds listed are also useful in the method of the disclosure and in pharmaceutical compositions of the disclosure.

In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a tablet, pill, capsule, powders, granules, gel, gel capsule, or cream. In such solid dosage forms, the active compound is admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia gum, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof.

Solid dosage forms can be prepared with coatings and shells, such as enteric coatings and others well known in the art. They may contain opacifying agents and can also be of such composition that they release the active compound or compounds in a certain part of the intestinal tract in a delayed manner. In certain embodiments, pharmaceutical composition is in solid form surrounded by an enteric coating. In certain embodiments, the enteric coating comprises methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, or combinations thereof

In certain embodiments, this disclosure contemplates an intravenous formulation with pH buffering agents and tonicity (isotonic) in a range representing physiological values (pH of about 7 to 8) or for bolus administration, e.g., containing normal saline or dextrose optionally containing pH buffering agents. In certain embodiments, the pharmaceutical composition is in the form of a sterilized pH buffered aqueous salt solution or a saline phosphate buffer between a pH of 6 to 8, optionally comprising a saccharide or polysaccharide.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

These compositions may also contain preserving, emulsifying, and dispensing agents. Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

In certain embodiments, this disclosure relates to kits comprising agents disclosed herein. The kits may contain a transfer device such a needle, syringe, cannula, capillary tube, pipette, or pipette tip. In certain embodiments, the agents may be contained in a storage container, sealed, or unsealed, such a vial, bottle, ampule, blister pack, or box. In certain embodiments, the kit further comprises written instructions for using the agents for treating and/or preventing a tumor or neoplasm in a subject, and/or increasing or lengthening survival of a subject having cancer, tumor, or neoplasm.

### Hypomorph Mutation-Directed Small-Molecule Protein-Protein Interaction Inducers to Restore Mutant SMAD4-Suppressed TGF-β Signaling

Tumor suppressor genes represent a major class of oncogenic drivers. However, direct targeting of loss-of-function tumor suppressors remains challenging. To address this gap, a variant-directed chemical biology approach was explored to reverse the lost function of tumor suppressors using SMAD4 as an example. SMAD4, a central mediator of the TGF-β pathway, is recurrently mutated in many tumors. Reported herein is a TR-FRET technology that recapitulated the dynamic differential interaction of SMAD4 and SMAD4R361H mutants with SMAD3 which is used to identify Ro-31-8220, a bisindolylmaleimide derivative, as a SMAD4R361H/SMAD3 interaction inducer. Ro-31-8220 reactivated the dormant SMAD4R361H-mediated transcriptional activity and restored TGF-β-induced tumor suppression activity in SMAD4 mutant cancer cells. This demonstrates that Ro-31-8220 as a SMAD4R361H/SMAD3 interaction inducer can be used as general strategy to reverse the lost function of tumor suppressors with hypomorph mutations and supports a systematic approach to develop small-molecule protein-protein interaction (PPI) molecular glues for therapeutic applications.

Reported herein is a general high-throughput screening approach to identify small molecules that can induce and stabilize the interaction of SMAD3 with mutated SMAD4 using a time-resolved fluorescence resonance energy transfer (TR-FRET) assay in a multi-well plate-based ultra-high-throughput screening (uHTS) format and the identification of compounds that can induce the interaction of mutated SMAD4 with SMAD3.

In certain embodiments, this disclosure relates to methods of screening a test compound for the ability to induce SMAD4 and another SMAD protein to interact comprising: contacting a SMAD4 protein conjugated to a first fluorophore with another SMAD protein conjugated to a second fluorophores in an aqueous solution, wherein the first and second fluorophores are donor acceptor pairs and measuring, detecting, or quantifying a background signal of the aqueous solution; contacting a test compound with the SMAD4 protein conjugated to the first fluorophore and with another SMAD protein conjugated to the second fluorophore, whereby the test compound induces the SMAD4 protein and the other SMAD protein to form a complex bringing the first and second fluorophores into close proximity for generating a binding signal that is different from the background signal and measuring, detecting, or quantifying a background signal.

In certain embodiments, methods further comprise recording the background signal and the binding signal on a computer readable medium. In certain embodiments, methods further comprise calculating a difference between the background signal and the binding signal and recording the difference on a computer readable medium.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general-purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### EXAMPLES

### Design and Development of the TR-FRET Assay for Identifying SMAD4/SMAD3 Protein-Protein Interaction (PPI) Inducers

SMAD4 R361H mutation, one of the hotspot missense mutations in the MH2 domain, impairs the protein-protein interaction (PPI) between SMAD4 and SMAD3, leading to the disruption of the transcriptional complex, and blocking TGF-b-induced anti-proliferation signaling (Fleming et al., 2013).

To enable the unbiased systematic discovery of small-molecule PPI inducers, a sensitive and scalable high-throughput technology platform was used to monitor the differential PPI status between SMAD3 and SMAD4 WT or R361H. The feasibility of the TR-FRET technology was examined and tested in a miniaturized multi-well plate format. Using the known SMAD4-SMAD3

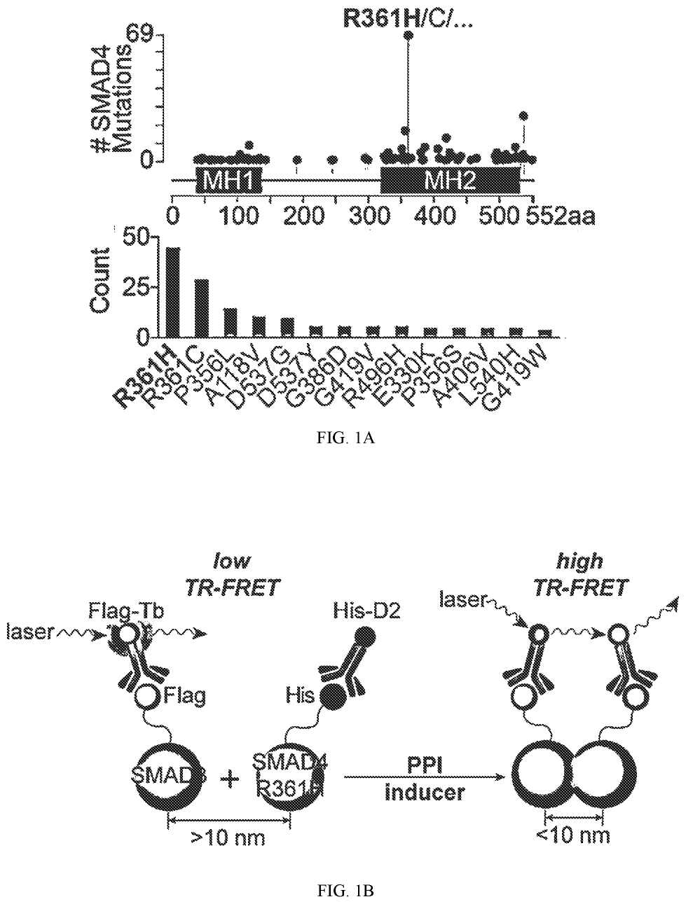

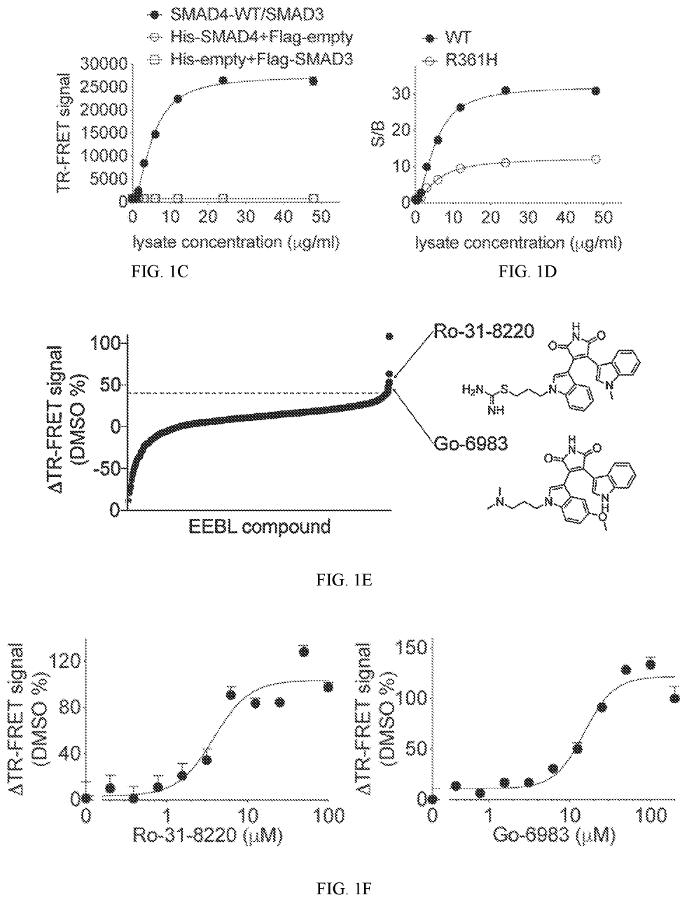

PPI as a positive control, the TR-FRET assay was optimized using cell lysates that co-expressed His-SMAD4 and Flag-SMAD3, and the coupled fluorophore pair anti-flag-Tb (donor) and anti-His-D2 (acceptor). The direct SMAD4-SMAD3 PPI brings Tb and D2 fluorophores into sufficiently close proximity generating a TR-FRET signals (FIG. 1B). The cell lysate containing His-SMAD4 and Flag-SMAD3 exhibited significantly higher TR-FRET signals than the corresponding empty vector controls in a concentration-dependent manner. These results suggested the feasibility of using this FRET donor/acceptor configuration to monitor the SMAD4-SMAD3 PPI.

To evaluate the sensitivity needed to capture a single amino acid change-induced difference of the SMAD4-SMAD3 PPI dynamics, the PPI signals between SMAD4 WT and R361H mutant were compared with SMAD3. With the same configuration of protein tags and fluorophore-conjugated antibody pairs, SMAD4R361H exhibited drastically decreased TR-FRET signals, showing a significant differential assay window along with the WT SMAD4 PPI (signal-to-background ratio of 10-fold versus 30-fold) (FIG. 1C). These results confirmed the importance of R361 residue of SMAD4 in heteromeric SMAD4/SMAD3 complex formation, and most importantly demonstrated the high sensitivity of the TR-FRET assay in monitoring the differential PPI dynamics at single-amino acid resolution.

### TR-FRET Sreening Reveals Potential PPI Inducers for Mutant SMAD4

To examine the utility of this TR-FRET assay to identify small-molecule PPI inducers that can restore mutant SMAD4 interaction with SMAD3, a library of 2,036 bioactive compounds were screened using the His-SMAD4R361H/Flag-SMAD3 pair. To maximize the opportunity to reveal inducers that can enhance mutant SMAD4/SMAD3 interaction, the EC90 (90% maximal effective concentration) condition of the SMAD4R361H/SMAD3 interaction was selected, which corresponded to EC10 (10% maximal effective concentration) of the WT SMAD4/SMAD3 PPI. This selection was based on our assessment of the assay performance, such as assay window between WT and R361H, DMSO tolerance, and robustness in the multi-well plate uHTS format.

The cell lysate co-expressing His-SMAD4R361H and Flag-SMAD3 were treated with the test compounds (20 μM). Based on the potentiation effect of >40% compared with the DMSO control, the top ranked 23 primary hits were picked for dose-response studies (FIG. 1D). Hits were validated with significant and reproducible effect in increasing the TR-FRET signal of SMAD4R361H-SMAD3 PPI.

### Identification of Ro-31-8220 and Go-6983 as SMAD4R361H-SMAD3 PPI Inducers

Among positive hit compounds, Ro-31-8220 and Go-6983, two bisindolylmaleimide compounds, showed robust potentiation effect of >40% for the SMAD4R361H-SMAD3 PPI from the primary screening. Ro-31-8220 and Go-6983 induced the increase of the TR-FRET signal in a dose-dependent manner with half maximal effective concentration (EC50) of 3.9±1.0 and 15.2±2.9 μM, respectively (FIG. 1E). These data confirmed their activity in promoting the SMAD4R361H-SMAD3 interaction, demonstrating the utility of the TR-FRET assay for systematic discovery of PPI inducers.

To further validate the PPI stabilization effect of these two compounds in orthogonal assays, experiments were performed to determine whether they can restore SMAD4R361H-SMAD3 complex formation using an affinity-based GST beads pull-down assay. In the GST pull-down assay, a substantial amount of SMAD3 was detected in complex with GST-SMAD4WT, whereas the R361H mutation of SMAD4 significantly decreased SMAD3 in the complex. Upon treatment of the cell lysate with Ro-31-8220, the amount of SMAD3 in the SMAD4R361H complex was significantly increased in a dose-dependent manner, with an EC50 of 2.1±0.7 μM. Similarly, Go-6983 enhanced the complex formation between SMAD3 and SMAD4R361H with an EC50 of 8.9±0.7 μM. These data support the potential stabilization effect of Ro-31-8220 and Go-6983 in enhancing the SMAD4R361H-SMAD3 interaction.

To evaluate the mutation selectivity of Ro-31-8220 and Go-6983, their potentiation effect on other SMAD4 loss-of-function mutations, such as D351H, was examined. The PPI between SMAD3 and SMAD4D351H was studied in the presence of these compounds with DMSO as a control. Similarly, D351H impairs SMAD4-SMAD3 PPI complex formation, and treatment with Ro-31-8220 and Go-6983 significantly restored the SMAD3 level in the SMAD3D351H GST pull-down complex. Altogether, the results suggest that these compounds can enhance the interaction between both D351H and R361H with SMAD4 and SMAD3. These data imply that reduced interaction with SMAD4 by selected mutant SMAD4 may be due to a shared mechanism.

### SMAD4 Loss-of-Function Mutations with other Receptor-Regulated SMADs (R-SMADs)

To determine the R-SMADs selectivity of Ro-31-8220 and Go-6983, experiments were performed to determine their effects on inducing PPI between SMAD4R361H and different isoforms of R-SMADs using the TR-FRET assay. With the compound titration, Ro-31-8220-induced increase of the TR-FRET signal is significantly higher for the SMAD4R361H interaction with SMAD3 than that with other R-SMADs. Similarly, Go-6983 induced a significantly higher PPI signal for SMAD4R361H interaction with SMAD3 as compared with other R-SMADs. Among these R-SMADs, SMAD3 and SMAD2, which mediate TGF-β signaling, appeared to have significantly higher compound-induced TR-FRET signals than SMAD1, SMAD5, and SMAD8, which mediate bone morphogenetic protein (BMP) signaling. These data suggest that, among all the tested R-SMADs, Ro-31-8220 and Go-6983 are selective in inducing the interaction of SMAD4R361H with SMAD3 and SMAD2.

### Other Bisindolylmaleimides

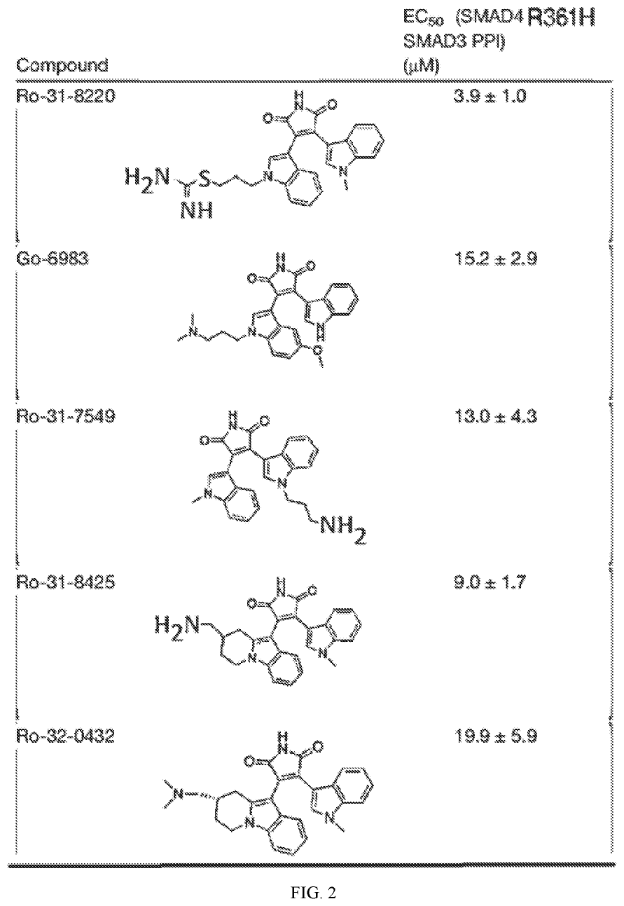

Ro-31-8220 and Go-6983, are structurally similar staurosporine analogs with a reported activity as ATP-competitive inhibitors of protein kinase C (PKC) (Davis et al., 1989). Given the in vitro cell lysate conditions used for the identification of Ro-31-8220 and Go-6983 as SMAD4R361H-SMAD3 PPI inducers, experiments were performed to determine whether such PPI stabilization effects are due to their PKC inhibitory activity. a panel of PKC inhibitors were selected, including five bisindolylmaleimide derivatives and two structurally diverse PKC inhibitors, and their activities were tested in enhancing the SMAD4R361H-SMAD3 PPI. From the dose-response study using the TR-FRET assay, all the bisindolylmaleimide derivatives exhibited similar PPI potentiation effects, with EC₅₀ values ranging from 4 to 20 μM, whereas the other two structurally diverse PKC inhibitors had no significant effect up to 100 μM (FIG. 2). Among these compounds, there is no significant correlation between their PPI-enhancing and PKC-inhibitory activities. Taken together, these results suggest that the SMAD4R361H-SMAD3 PPI stabilization effect induced by Ro-31-8220 is most likely due to the bisindolylmaleimide core structure.

### Ro-31-8220 restores TGF-β Tumor Suppression Function in SMAD4 Mutant Colon Cancer cells

Experiments indicate that Ro-31-8220 is capable of restoring the SMAD4mut/SMAD3 PPI and the tumor-suppressive TGF-β signaling in the SMAD4 mutation cells. Experiments were also performed to determine whether Ro-31-8220 can promote TGF-β-evoked anti-proliferation function in cancer cells with SMAD4 missense mutations. Three functional assays were performed to measure the effect of Ro-31-8220 on cell-cycle progression, cell proliferation, and colony formation with WT and isogenic SMAD4 mutant colon cancer cells.

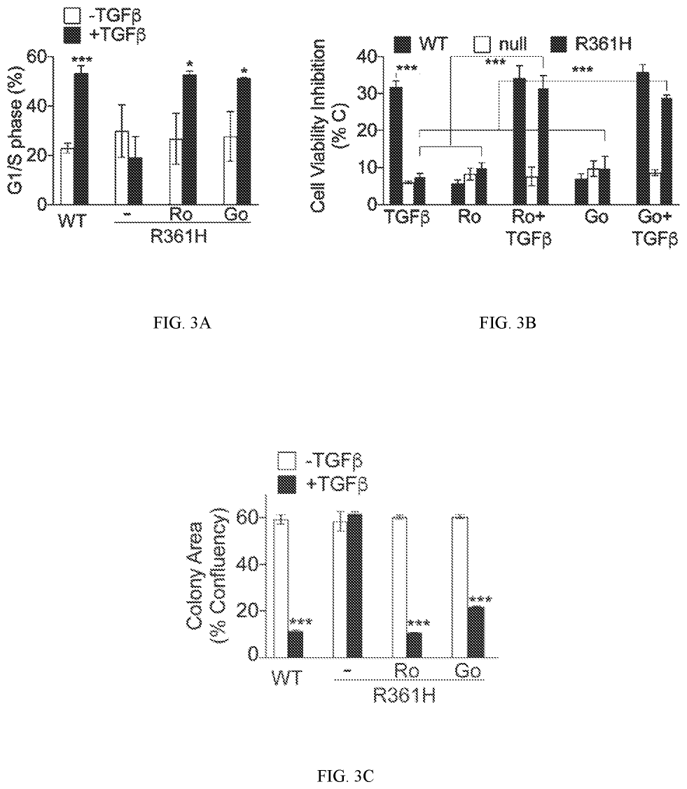

From flow cytometry-based cell-cycle analysis, WT cells were sensitive to TGF-β, showing significantly increased number of cells in the G1/S phase, while the R361H isogenic cells were insensitive to TGF-β-induced G1/S arrest (FIG. 3A), which is consistent with the cell-cycle-associated gene expression signatures. However, R361H cells treated with Ro-31-8220 regained sensitivity to TGF-β-induced G1/S cell-cycle arrest. A similar re-sensitization effect was also observed with Go-6983 treatment. These results support the role of Ro-31-8220 in restoring the SMAD4R361H/SMAD3 PPI to transmit TGF-β-triggered signaling, resulting in expression of defined cell-cycle regulatory genes and the enhanced cell-cycle arrest.

As well as the cell-cycle regulatory genes, Ro-31-8220 treatment also restored the transcriptional response of R361H cells to TGF-β in terms of pro-apoptotic and pro-autophagy genes that may promote cell death. Thus, the effect of Ro-31-8220 on TGF-β-regulated cell proliferation was examined in the context of the SMAD4 mutation. TGF-β induced a significant decrease of cell viability of SMAD4 WT cells, while the isogenic SMAD4 null or R361H cells were resistant to such TGF-β-induced growth suppression (FIG. 3B). The Ro-31-8220 treatment alone did not result in any significant growth inhibition among SMAD4 WT, null, and R361H cells. However, Ro-31-8220 restored the sensitivity of SMAD4 R361H cells, but not null cells, to TGF-β-induced growth suppression, showing significantly reduced viability comparable with that of WT cells (FIG. 3B). The combination of Ro-31-8220 with TGF-β exhibited a synergistic effect, showing enhanced growth inhibition of cells with SMAD4 R361H. Similar effects were also observed for Go-6983 in this set of isogenic cells, as well as in a panel of patient-derived colon cells (FIGS. 3B).

The lost tumor suppressor function of mutated SMAD4, R361H, can be recapitulated in an in vitro tumorigenic assay. Colon cancer HCT116 cells with WT and their isogenic mutant SMAD4 cells can grow into large colonies. This clonogenic activity of HCT116 with SMAD4WT can be suppressed by TGF-β showing diminished sizes of the colonies formed, whereas isogenic cells with SMAD4 R361H were resistant to TGF-β, retainingβ, the R361H cells showed significantly decreased colony area, suggesting that they regained the sensitivity to TGF-β-induced inhibition of colony formation. A similar effect was also observed for Go-6983 in this pair of isogenic cell lines, as well as in other patient-derived colon cancer cells with SMAD4 mutations. Altogether, our results suggest that Ro-31-8220 and Go-6983 can synergize with TGF-62 to induce the growth inhibition and clonogenic activity of colon cancer cells harboring the SMAD4 missense mutations, and such a synergistic effect depends on the presence of mutant SMAD4 proteins.

1. A method of diagnosing and treating cancer comprising
obtaining a sample from a subject and detecting a mutation of tumor suppressor gene SMAD4 in the sample encoding an alternate amino; and
administering an effective amount of a compound having a 3,4-di(indol-3-yl)-pyrrole-2,5-dione backbone structure, or salt thereof, to the subject in need thereof.

2. The method of claim 1, wherein the mutation encoding the alternate amino acid is at position 361.

3. The method of claim 1, wherein the mutation encoding amino acid position 361 of tumor suppressor gene SMAD4 is a codon mutation that translates histidine (H) or cysteine (C) instead of arginine (R) at amino acid position 361.

4. The method of claim 1, wherein the subject is diagnosed with colon cancer, pancreatic cancer, skin cancer, stomach cancer, esophageal cancer, cervical cancer, or lung cancer.

5. The method of claim 1, wherein the compound having a 3,4-di(indol-3-yl)-pyrrole-2,5-dione backbone structure is administered in combination with another anticancer agent.

6. The method of claim 1, wherein the compound is 3-(3-(4-(1-methyl-1H-indol-3-yl)-2,5-dioxo-2,5-dihydro-1H-pyrrol-3-yl)-1H-indol-1-yl)propyl carbamimidothioate (Ro-31-8220), salt, or derivative thereof.

7. The method of claim 1, wherein the compound is 3-(1-(3-(dimethylamino)propyl)-5-methoxy-1H-indol-3-yl)-4-(1H-indol-3 -yl)-1H-pyrrole-2,5-dione (Go-6983), salt, or derivative thereof.

8. The method of claim 1, wherein the compound is 3-(1-(3-aminopropyl)-1H-indol-3-yl) (1-methyl-1H-indol-3-yl)-1H-pyrrole-2,5-dione (Ro-31-7549), salt, or derivative thereof.

9. The method of claim 1, wherein the compound is 3-(8-(aminomethyl)-6,7,8,9-tetrahydropyrido[1,2-a]indol-10-yl)-4-(1-methyl-1H-indol-3-yl)-1H-pyrrole-2,5-dione (Ro-31-8425), salt, or derivative thereof.

10. The method of claim 1, wherein the compound is 3-(8-((dimethylamino)methyl)-6,7,8,9-tetrahydropyrido[1,2-a]indol-10-yl)-4-(1-methyl-1H-indol-3-yl)-1H-pyrrole-2,5-dione (Ro-32-0432), salt, or derivative thereof.

11. A method of treating cancer comprising administering an effective amount of a compound having a 3,4-di(indol-3-yl)-pyrrole-2,5-dione backbone structure, or salt thereof, to the subject in need thereof;
wherein the compound is selected from
3-(3-(4-(1-methyl-1H-indol-3-yl)-2,5-dioxo-2,5-dihydro-1H-pyrrol-3-yl)-1H-indol-1-yl)propyl carbamimidothioate (Ro-31-8220);
3-(1-(3-(dimethylamino)propyl)-5-methoxy-1H-indol-3-yl)-4-(1H-indol-3-yl)-1H-pyrrole-2,5-dione (Go-6983);
3-(1-(3-aminopropyl)-1H-indol-3-yl)-4-(1-methyl-1H-indol-3-yl)-1H-pyrrole-2,5-dione (Ro-31-7549);
3-(8-(aminomethyl)-6,7,8,9-tetrahydropyrido[1,2-a]indol-10-yl)-4-(1-methyl-1H-indol-3-yl)-1H-pyrrole-2,5-dione (Ro-31-8425); and
3-(8-((dimethylamino)methyl)-6,7,8,9-tetrahydropyrido[1,2-a]indol-10-yl)-4-(1-methyl-1H-indol-3-yl)-1H-pyrrole-2,5-dione (Ro-32-0432), salts, or derivatives thereof.

12. The method of claim 11, wherein the cancer is solid tumor.

13. The method of claim 11, wherein the cancer is colon cancer.

14. The method of claim 11, wherein the cancer is pancreatic cancer.

15. The method of claim 11, wherein the cancer is skin cancer.

16. The method of claim 11, wherein the subject is experiencing recurrence of the cancer.

17. A method of screening a test compound for the ability to induce SMAD4 and another SMAD protein to interact comprising:
contacting a SMAD4 protein conjugated to a first fluorophore with another SMAD protein conjugated to a second fluorophores in an aqueous solution, wherein the first and second fluorophores are donor acceptor pairs and measuring, detecting, or quantifying a background signal of the aqueous solution;
contacting a test compound with the SMAD4 protein conjugated to the first fluorophore and with another SMAD protein conjugated to the second fluorophore, whereby the test compound induces the SMAD4 protein and the other SMAD protein to form a complex bringing the first and second fluorophores into close proximity for generating a binding signal that is different from the background signal and measuring, detecting, or quantifying a background signal.

18. The method of claim 17, further comprising recording the background signal and the binding signal on a computer readable medium.

19. The method of claim 18, further comprising calculating a difference between the background signal and the binding signal and recording the difference on a computer readable medium.
